(19) United States
(12) Patent Application Publication
(10) Pub. No.: US 2026/0260760 A1
(43) Pub. Date: Sep. 3, 2026

(54) TRAINING AND APPLICATION OF ARTIFICIAL INTELLIGENCE MODELS FOR PREDICTING THE OCCURRENCE OF CANCER IN AN INDIVIDUAL

(71) Applicant: HUNA LTDA., São Paulo (BR)

(72) Inventors: Vinicius MOURA RIBEIRO, Laranjeiras, Rio de Janeiro (BR); Daniella CASTRO ARAUJO, Goiânia (BR); Marco Aurélio KOHARA, São Paulo (BR)

(21) Appl. No.: 19/164,568

(22) PCT Filed: Mar. 22, 2024
(86) PCT No.: PCT/BR2024/050106

§ 371 Date: Sep. 11, 2025

(30) Foreign Application Priority Data
Mar. 23, 2023 (BR) 1020230053912

(51) Int. Cl. G16H 50/30; G06N 20/00

(52) U.S. Cl. CPC G16H 50/30; G06N 20/00

(57) ABSTRACT

The present invention relates to methods and systems for training an artificial intelligence model to predict the chances of cancer occurring in an individual based on features obtainable from a routine blood test. The present invention also relates to methods and systems for predicting the chances of cancer occurring in an individual based on the trained artificial intelligence model.

### TECHNICAL FIELD

The invention relates to the technical field of artificial intelligence and, in particular, to methods and systems for assessing the risk of cancer occurring in an individual based on features obtained in a routine blood test.

### PRIOR ART

Cancer is a disease caused by the disorderly multiplication of abnormal cells that form a tumor with the potential to invade certain organs. Some types of cancer develop rapidly, while others grow slowly. In most cases, when cancer is treated appropriately and in a timely manner, there is good prognosis for a cure.

One of the most promising technologies for early cancer detection is liquid biopsies, which generally detect circulating pools of tumor-associated cells, or circulating cell-free DNA, through specific blood tests. However, this technology remains complex and expensive, making its widespread implementation unfeasible. Other existing techniques rely on mammogram results. However, mammograms are also not widespread worldwide, especially in poorer countries.

Therefore, there is still a need for new technologies, especially those that are low-cost and based on easily obtainable data, capable of achieving satisfactory prediction rates of cancer occurrence in an individual. Additionally, such technologies would allow for the stratification of cancer risk in a population, enabling more personalized screening, particularly useful in low-income places that prevent broader and more complex screenings.

### SUMMARY OF THE INVENTION

The present invention relates to a method for training an artificial intelligence model to predict the chances of a solid tumor occurring in an individual, the method comprising at least the steps of: receiving a first set of features, wherein the first set of features is related to individuals who have not been diagnosed with some type of solid tumor and comprises, as features for each individual, at least: age; at least one feature obtained from the red blood cell series of a blood count; and at least one feature obtained from the white blood cell series of a blood count; receiving a second set of features, in which the second set of features is related to individuals diagnosed with some type of solid tumor and comprises, as features for each individual, at least: age; at least one feature obtained from the red blood cell series of a blood count; and at least one feature obtained from the white blood cell series of a blood count; and training the artificial intelligence model based on the first and second sets of features.

Optionally, the solid tumor to be predicted is selected from the group consisting of: breast cancer, lung cancer, colorectal cancer, prostate cancer, and ovarian cancer.

Optionally, the solid tumor is breast cancer.

Optionally, the first and second sets of features additionally comprise at least one feature obtained from the platelet series of a blood count.

Optionally, the first and second sets of features additionally comprise at least one feature obtained from the individual's phenotypic characteristics and/or from imaging tests or routine medical examinations.

Optionally, the model is trained using a supervised or unsupervised machine learning algorithm, or comprises the combination of models trained using a supervised and/or unsupervised learning algorithm.

The present invention also relates to a system for training an artificial intelligence model to predict the chances of a solid tumor occurring in an individual, the system comprising at least one processor, wherein the processor is configured to perform the method for training an artificial intelligence model as described above.

The present invention also relates to a computer-readable medium comprising instructions that, when executed by at least one processor, cause the processor to perform the method for training an artificial intelligence model as described above.

The present invention also relates to a method for predicting the chances of a solid tumor occurring in an individual, comprising the steps of: receiving, as features for the individual, at least: age; at least one feature obtained from the red blood cell series of a blood count; and at least one feature obtained from the white blood cell series of a blood count; and calculating, using a model trained according to the method for training an artificial intelligence model as described above and based on the received features, the chances of cancer occurring in the individual.

Optionally, the solid tumor to be predicted is selected from the group consisting of: breast cancer, lung cancer, colorectal cancer, prostate cancer, and ovarian cancer.

Optionally, the solid tumor is breast cancer.

Optionally, the first and second sets of features additionally comprise at least one feature obtained from the platelet series of a blood count.

Optionally, the first and second sets of features additionally comprise at least one feature obtained from the individual's phenotypic characteristics and/or from imaging tests or routine medical examinations.

The present invention also relates to a system for predicting the chances of a solid tumor occurring in an individual, the system comprising at least one processor, wherein the processor is configured to perform the method for predicting the chances of a solid tumor occurring in an individual as described above.

The present invention also relates to a computer-readable medium comprising instructions that, when executed by at least one processor, cause the processor to perform the method for predicting the chances of a solid tumor occurring in an individual as described above.

### BRIEF DESCRIPTION OF THE FIGURES

FIG. 1 illustrates an exemplary architecture of a system according to the present invention.

FIG. 2 illustrates a SHAP graph showing the effect of each feature on the results obtained by the model according to a first embodiment of the present invention.

FIG. 3 illustrates a graph showing the effect of each feature as a Ridge coefficient in a model according to a second embodiment of the present invention.

FIG. 4 illustrates the results obtained with a model of the second embodiment of the present invention for different types of tumors.

FIG. 5 illustrates a table of correlations between features of the white blood cell and platelet series that can be used in the first embodiment of the present invention.

FIG. 6 illustrates a table of correlations between features of the red blood cell series that can be used in the first embodiment of the present invention.

### DETAILED DESCRIPTION

The present invention relates to artificial intelligence techniques applied to predicting the occurrence of cancer in an individual. The inventors discovered that, based on features obtainable from routine blood tests (full blood count—FBC), it is possible to create and apply models with an adequate accuracy rate in predicting the occurrence of solid tumors.

FBC is commonly divided into three groups: white blood cell count or series (leukogram); red blood cell count or series (erythrogram); and platelet count or series (platelet count). The red blood cell series evaluates the parameters related to red blood cells. The white blood cell series evaluates the different types of white blood cells. The platelet series is primarily intended for counting and analyzing platelets.

Within the context of the present invention, a feature obtainable from a routine blood test is a feature that comprises one or more values obtained from the red blood cell series, the white blood cell series, and/or the platelet series. The red series is the group that includes, for example, values for: hemoglobin, red blood cell count (RBC), hematocrit, mean corpuscular volume (MCV), mean corpuscular hemoglobin (MCH), mean corpuscular hemoglobin concentration (MCHC), and red blood cell distribution width (RDW). The white series is the group that includes, for example, values for: leukocytes, neutrophils, rods, basophils, eosinophils, lymphocytes, monocytes, and blasts. The platelet series is the group that includes, for example, values for: platelets and mean platelet volume (MPV).

Logically, features can be obtained from the combination of features (e.g., ratio or any formula that combines features) within the same series and from features between different series. Examples include: aggregated systemic inflammation index (neutrophils×monocytes×(platelets/lymphocytes)) (AISI), derived neutrophil-to-leukocyte ratio (neutrophils/(lymphocytes-neutrophils)) (dNLR), lymphocyte-to-monocyte ratio (LMR), neutrophil-to-lymphocyte ratio (NLR), platelet-to-lymphocyte ratio (PLR), systemic immune inflammation index (platelets×(neutrophils/lymphocytes)) (SII), systemic inflammation response index (neutrophils×(monocytes/lymphocytes)) (SIRI).

In the context of the present invention, the group of features obtainable in routine blood tests may also include an individual's age, which is usually provided by the individual when performing such tests or can be obtained directly from the individual or from some record of that individual, and any relationship between age and any other feature. All these features are easily obtained, without requiring the use of any complex and costly techniques or equipment.

It should be understood that, in addition to these features above, other features, even if not obtainable from routine blood tests, can be added for training and use of the models. Examples include features obtained from imaging tests, features related to an individual's phenotypic characteristics, and features obtained from routine medical examinations, such as in-office exams. Another example of another feature is breast density.

A model can be trained generically using two sets of features: features obtained from individuals diagnosed with cancer or who developed cancer after a certain period of time from the blood draw; features obtained from individuals who were not diagnosed with cancer and who did not develop cancer after a certain period of time from the blood draw.

To train a model, known machine learning techniques can be employed, such as supervised and unsupervised learning techniques. Examples of supervised learning techniques include decision trees, k-Nearest Neighbors algorithms, Naive Bayes, and different types of regression. Examples of unsupervised learning techniques include genetic algorithms, neural networks, Support Vector Machines, and regression and fuzzy algorithms.

In the context of the present invention, a model can also comprise a combination of models trained using different techniques. This combination is known in the prior art as an ensemble.

Once trained, the model acts as a classifier, receiving features as input and outputting the prediction of the occurrence of cancer in an individual.

For training a model and for using a trained model, the features may or may not be encoded. In the absence of encoding, features such as those measured and reported according to the standards applied in routine blood tests may be used. An example of encoding is the application of some normalization technique to the input features.

In a preferred embodiment of the present invention, an individual's age, at least one feature obtained from the red blood cell series of a blood count, and at least one feature obtained from the white blood cell series of a blood count are used as features for training and using a model.

In another preferred embodiment of the present invention, an individual's age, at least one feature obtained from the red blood cell series of a blood count, at least one feature obtained from the white blood cell series of a blood count, and at least one feature obtained from the platelet series of a blood count are used as features for training and using a model.

The tumor to be predicted is a solid tumor. Examples include breast cancer, lung cancer, colorectal cancer, prostate cancer, and ovarian cancer.

In a preferred embodiment of the present invention, the cancer to be predicted by the model is breast cancer.

A computer-readable medium is any medium capable of being accessed by a processor and capable of storing instructions that can be read by the processor for the execution of a method. Examples include flash drives, external and internal hard drives, CD-ROMs, and RAM and ROM memories.

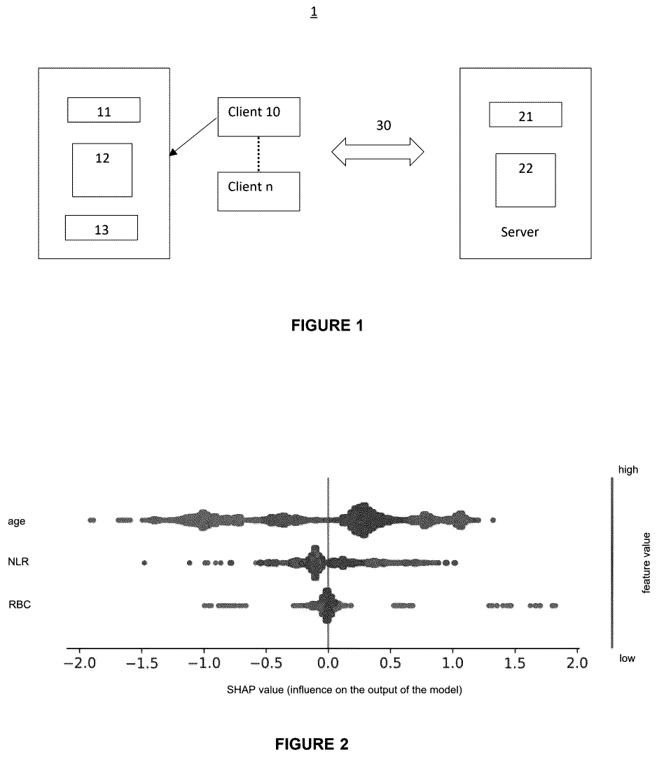

FIG. 1 illustrates an example of a client/server system 1 that can be implemented for the purposes of the present invention. As can be seen, one or more client devices 10-n are connected to a server and communicate through an interface 30.

The client devices 10-n include one or more processors 11, a memory set 12, and data input and output interfaces 13, and can be implemented as personal devices or server terminals, taking the form, for example, of laptops, desktops, tablets, or smartphones.

The data input and output interfaces 13 may be, for example, a keyboard, a mouse, a microphone, a speaker, and a touchscreen.

The interface 30 may be a wired or wireless communication interface, such as a WAN, LAN, or the Internet.

The server includes one or more processors 21 and a set of memories 22 and may be implemented as a personal computer, a network server, or a virtual server.

The set of memories 12 and 22 may include transient and non-transitory computer-readable media, such as ROM and RAM. The set of memories 12 and 22 typically store both the operating systems of the client and server devices and computer-readable instructions for training and using models based on the features of one or more individuals.

In the exemplary system 1, a model may be trained by one or more processors 21 of the server and also stored in the set of memories 22 of the server. Alternatively, the model may be trained on another processing device, such as a client device, and stored in the in the set of memories 22 of the server. Alternatively, the model may be trained by the server and one or more client devices jointly.

With a trained model, a client device 1-n may, through the data input and output interfaces 13, receive and send to the server features associated with an individual. Upon receiving the features, the server processor 21 uses them as input to the trained model, which estimates the chances of cancer occurring for the individual associated with those features. These chances of occurrence are made available to the client device 1-n in return. Alternatively, a client device 1-n may receive and send to the server features of more than one individual.

Likewise, the server receives and processes these features, returning the chances of cancer occurrence for all individuals associated with such features.

As anyone skilled in the art will appreciate, system 1 in FIG. 1 does not need to be implemented in a client/server architecture. In this situation, there is no server. The processor and set of memories of a client device, such as a personal computer, are responsible for training and/or storing the model and executing the instructions for calculating the chances of cancer occurrence in an individual.

### EXAMPLES OF EMBODIMENTS

To train the models used in the embodiment examples of the present invention below, two supervised machine learning techniques were employed. Two models were trained using ridge regression, as described in “Hoerl, A. E. & Kennard, R. W. Ridge regression: Biased estimation for nonorthogonal problems. Technometrics 12, 55 (1970).” Two models were trained using the Gradient Boosting (GBM) technique, using the LightGBM algorithm described in “Guyon I, Von Luxburg U, Bengio S, Wallach H, Fergus R, Vishwanathan S, et al. LightGBM: A Highly Efficient Gradient Boosting Decision Tree. Von Luxburg and S. Bengio and H. Wallach and R. Fergus and S. Vishwanathan and R. Garnett IGAU, editor. Curran Associates, Inc.; 2017.”

In addition to age, the following features obtainable from a blood test were tested: eosinophils; hematocrit; hemoglobin; leukocytes; lymphocytes; MCH; MCHC; MCV; monocytes; neutrophils; platelets; RBC and RDW. The following derived features were also tested: AISI; dNLR; LMR; NLR; PLR; SII and SIRI.

The performance of each generated model was evaluated using the AUC (area under the curve) measure as described in “Fawcett T. in an introduction to ROC analysis. Pattern Recognition Letters. 2006. pp. 861-874. doi:10.1016/j.patrec.2005.10.010”. AUC performance was obtained by plotting the true positive rate as a function of the false positive rate at varying thresholds. Since the model output is a probability, i.e., the risk of cancer occurrence, the threshold ranges from 0 to 1. In each performance evaluation, a 95% confidence interval (CI) is predicted, ensuring statistical significance. This was achieved by performing 1,000 bootstrap resamplings with repositioning of the training set.

The features were normalized based on the maximum and minimum values of each feature in the training set to ensure uniformity across scale. To prevent data leakage, feature selection and hyperparameter tuning were performed only on the training set. The test set was reserved for the final evaluation.

For feature evaluation, a selection method guided by a directed acyclic graph was used. This approach begins with the selection of a single feature that provides the highest AUC value. Then, two features are selected, again based on the highest AUC value. The process continues by adding one new feature at a time and ends when the increase in the AUC value no longer exceeds the standard deviation, ensuring a balance between performance and model complexity.

For the performance analysis of the Lightgbm model, the SHAP (SHapley Additive exPlanations) methodology was used, as described in “Berry, R. F. & Hellerstein, J. L. A unified approach to interpreting measurement data in performance management applications. Proceedings of 1993 IEEE 1st International Workshop on Systems Management Preprint.” The SHAP methodology evaluates the importance of each feature by measuring the impact of its omission on the model output. It produces SHAP values for each input, indicating the importance of each feature to the model.

### Example 1—Prediction of Breast Cancer Occurrence

In this first example of implementation, the prediction of breast cancer occurrence in women was studied. To this end, blood test results from 396,848 women aged between 40 and 70 years old who were tested for breast cancer between January 2004 and August 2022 were collected from the Fleury Laboratory database.

The determination of breast cancer occurrence was based on carcinoma confirmations from histopathological reports or from Breast Imaging Reporting and Data System (BI-RADS) categories obtained from breast imaging reports (mammography, ultrasound, or magnetic resonance imaging). Thus, the case group comprises women who had breast cancer confirmed by a pathological examination or by a BI-RADS 5 (probability of breast cancer >95%), and who underwent a blood test within six months prior to the biopsy or imaging examination. The control group comprises women who did not receive a breast cancer confirmation from pathological examination and women with negative imaging examinations (BI-RADS 1 or 2).

The training set for the models includes: 979 BI-RADS 5 cases, each with a blood test performed within six months prior to the imaging examination; and 339,420 control group cases of women with BI-RADS 1 or 2, each with a blood test performed within six months prior to the imaging examination. The training set was further segmented into 80% for the training phase and 20% for the validation phase.

The test set includes: 1,882 cases with pathologically confirmed breast cancer, with a blood test performed within six months before the biopsy; 54,567 cases in the control group, comprising women who remained cancer-free for a period of 4.5 to 6.5 years, with at least three negative imaging tests (BI-RADS 1 or 2) within this period spaced at least 18 months apart, and with blood tests performed within a 6-month window centered on the date of each imaging test. For women with multiple blood tests in each window, only the first blood test was considered.

Of the 1,882 women diagnosed with breast cancer, 5% did not have information on the type of breast cancer (in situ vs. invasive) in the pathological report. Of the remaining 1,794, 437 had in situ cancer and 1,357 had invasive cancer.

Regarding molecular subtypes, of the 1,357 women with invasive breast cancer, 774 also underwent immunohistochemical testing. 532 women had luminal cancer, 170 had HER2-positive luminal cancer, 29 had HER2-enriched cancer, and 43 had triple-negative (TN) cancer.

Regarding T stage, invasive tumors were classified into three groups based on tumor size. The T stage could not be determined in 11% of invasive tumors. Of the remaining 1,209 women, 868 had cancers less than or equal to 2 cm (T1); 282 had cancers greater than 2 cm and less than or equal to 5 cm (T2); and 59 had cancers greater than 5 cm (T3).

Given the low prevalence of breast cancer in the general population, a significant imbalance was observed in the target variable in the training and test sets—0.29% and 3.3%, respectively. To address this imbalance, higher weights were applied to the cases for training both models.

### Example 1—Feature Analysis

Table 1 shows linear descriptive statistics for each feature. P-values were calculated using Student's t-test. P-values≤0.002 were considered significant (alpha was corrected from 0.05 to 0.001 using the Bonferroni test). Predictive AUCs were calculated by training a two-level decision tree on the training set and evaluating it on the test set.

TABLE 1

| | Mean - Control | Mean - Case | | |
|---|---|---|---|---|
| Feature | group | group | P value | Predictive AUC |
| Age (years) | 50.04 ± 8.56 | 53.92 ± 8.50 | 0.000 | 0.60 (0.58-0.61) |
| Eosinophils | 173.62 ± 149.65 | 170.59 ± 140.29 | 0.281 | 0.50 (0.49-0.51) |
| (/mm³) | | | | |
| Hematocrits (%) | 39.84 ± 2.96 | 40.02 ± 3.18 | 0.002 | 0.51 (0.50-0.52) |
| Hemoglobin | 13.23 ± 1.09 | 13.31 ± 1.15 | 0.000 | 0.50 (0.49-0.52) |
| (g/dL) | | | | |
| Leukocytes | 6421.13 ± 1894.44 | 6344.95 ± 1889.42 | 0.032 | 0.50 (0.50-0.51) |
| (/mm³) | | | | |
| Lymphocytes | 2079.67 ± 670.97 | 2000.46 ± 648.53 | 0.000 | 0.51 (0.50-0.51) |
| (/mm³) | | | | |
| MCH (pg) | 29.47 ± 2.08 | 29.66 ± 2.05 | 0.000 | 0.50 (0.49-0.51) |
| MCHC (%) | 33.19 ± 1.04 | 33.25 ± 0.98 | 0.005 | 0.50 (0.49-0.50) |
| MCV (fL) | 88.75 ± 5.19 | 89.19 ± 5.21 | 0.000 | 0.50 (0.50-0.51) |
| Monocytes | 479.78 ± 155.22 | 480.73 ± 164.89 | 0.744 | 0.50 (0.50-0.51) |
| (/mm³) | | | | |
| Neutrophils | 3650.25 ± 1468.55 | 3654.07 ± 1483.28 | 0.890 | 0.50 (0.49-0.50) |
| (/mm³) | | | | |
| Platelets | 256.8 ± 57.2 | 261.9 ± 61.1 | 0.540 | 0.50 (0.49-0.52) |
| (10³/mm³) | | | | |
| RBC (10⁶/mm³) | 4.50 ± 0.37 | 4.50 ± 0.40 | 0.831 | 0.51 (0.50-0.52) |
| RDW (%) | 13.33 ± 1.14 | 13.36 ± 1.14 | 0.067 | 0.48 (0.47-0.50) |
| AISI (×10⁶) | 236.8 ± 196.7 | 252.3 ± 219.4 | 0.000 | 0.51 (0.50-0.52) |
| dnlR | 1.37 ± 0.57 | 1.42 ± 0.62 | 0.000 | 0.51 (0.50-0.52) |
| LMR | 4.60 ± 2.03 | 4.45 ± 1.64 | 0.000 | 0.51 (0.50-0.51) |
| NLR | 1.87 ± 0.90 | 1.97 ± 1.01 | 0.000 | 0.51 (0.50-0.52) |
| PLR | 136.11 ± 47.12 | 143.06 ± 53.81 | 0.000 | 0.52 (0.50-0.53) |
| SII (×10³) | 481.4 ± 262.7 | 509.2 ± 287.6 | 0.000 | 0.51 (0.50-0.52) |
| SIRI | 921.53 ± 725.26 | 979.91 ± 836.79 | 0.000 | 0.51 (0.50-0.51) |

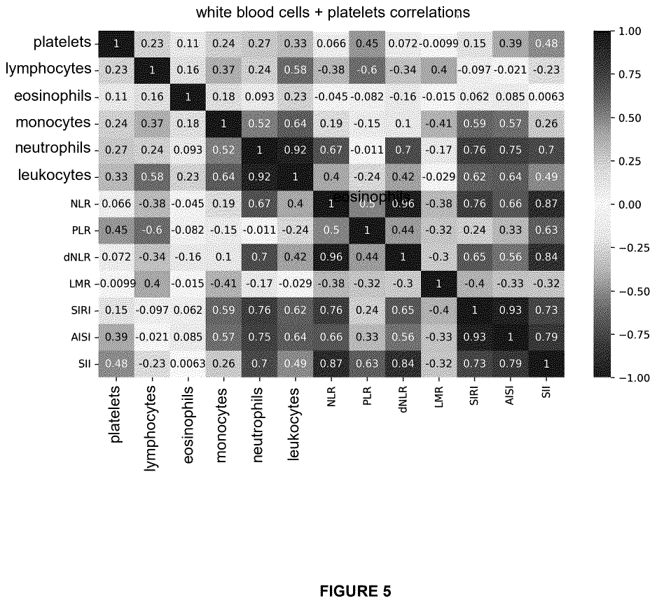

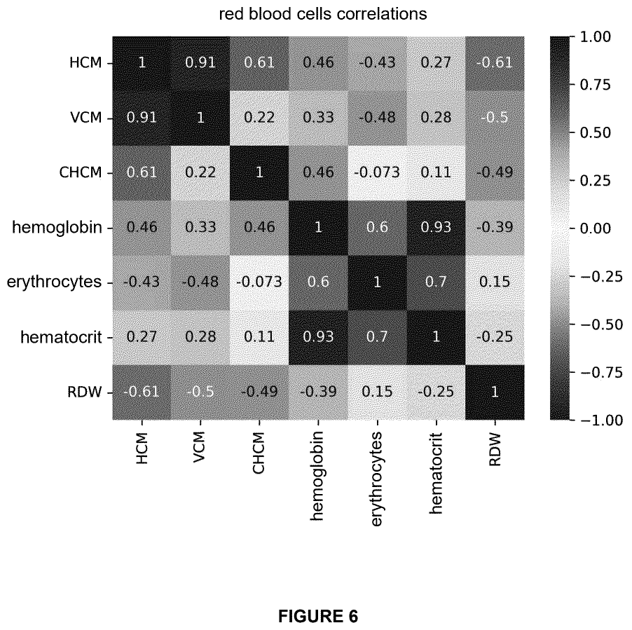

FIGS. 5 and 6 also show the correlation between the features of the white series and platelet series, and of the red series, respectively. Correlations greater than 0.40 can be considered moderate to strong. This indicates that replacing one feature with another moderately or strongly correlated feature does not significantly alter the model's AUC result.

### Example 1—LiqhtGBM Model

In this first example, two models were built using LightGBM: one model considering all the features in Table 1; and one model considering NLR, RBC, and age as features. The AUC performance of these models is shown in Table 2.

TABLE 2

| | | AUC - validation set | AUC - test set |
|---|---|---|---|
| Model | Features | (95% IC) | (95% IC) |
| 1 | All | 0.64 (0.61-0.66) | 0.62 (0.61-0.64) |
| 2 | NLR, RBC and age | 0.65 (0.63-0.67) | 0.63 (0.62-0.64) |

FIG. 2 illustrates the NLR, RBC, and age features in order of importance. The pink, purple, and blue dots represent higher, intermediate, and lower values, respectively. A vertical line separates women based on the model's probability, with positions to the right indicating a higher predicted cancer risk. Obviously, although the features are analyzed individually, the models consider non-obvious interactions between them.

As can be seen, for the age and NLR features, there is a predominance of blue dots on the left. The dots become purple and blue toward the right. This indicates that higher values of these features are related to a higher cancer risk. Conversely, the pattern observed for the RBC feature shows that lower values of this feature indicate a higher cancer risk.

### Example 1—Ridge Regression

Still in the first example of implementation, two models were built using Ridge regression: one model considering all the features in Table 1; and one model considering NLR, RBC, and age as features. The AUC performance of these models is shown in Table 3.

TABLE 3

| | | AUC - validation set | AUC - test set |
|---|---|---|---|
| Model | Features | (95% IC) | (95% IC) |
| 1 | All | 0.65 (0.64-0.66) | 0.63 (0.62-0.64) |
| 2 | NLR, RBC and age | 0.66 (0.65-0.66) | 0.64 (0.64-0.65) |

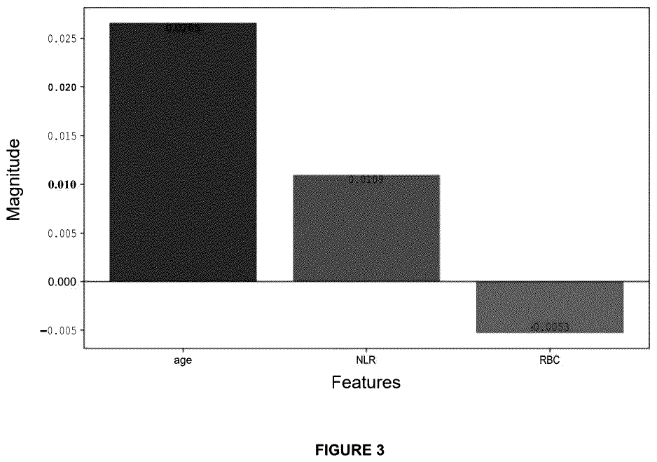

FIG. 3 illustrates the NLR, RBC, and age features in order of importance. In the Ridge regression model, higher age and NLR values are linked to a higher risk of breast cancer, while higher RBC values are associated with a lower risk. This is the same pattern observed in the LightGBM model.

### Example 2—Ridge Regression on Breast Tumors with Different Characteristics

In this second example, model 2 (Ridge regression) from example 1 was chosen, but trained on subsets of the database divided according to different tumor characteristics: invasive; in situ; luminal; HER2+; TN; ≤5 cm; and >5 cm.

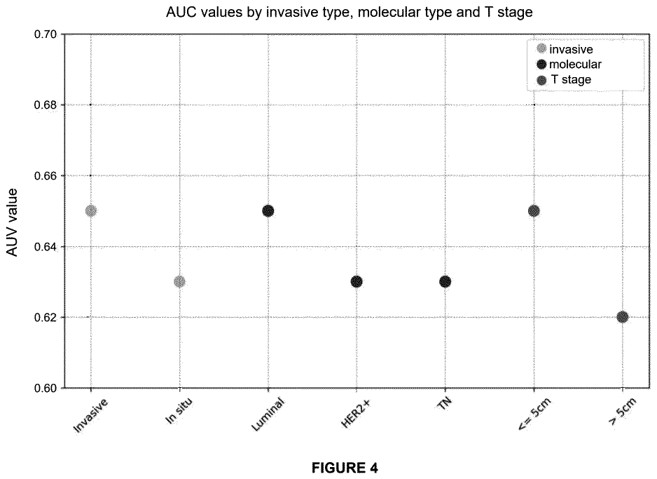

FIG. 4 illustrates the AUC result for each subset. The model in example 3 demonstrates superior performance (0.65) for invasive tumors compared to in situ tumors (0.63). In terms of molecular subtype, the model is more effective in predicting luminal tumors (0.65) than triple-negative and HER2+ tumors (0.63). Regarding T stage, the model demonstrates better results for smaller tumors (0.65) than for larger tumors (0.62).

### Example 3—Predicting Lung Cancer Occurrence

In this third implementation example, the same models from example 1, trained on the breast cancer database and with NLR, RBC, and age as features, were tested to predict lung cancer occurrence. To this end, blood test results from 291 female smokers aged 40 to 75 were collected from the HAmor database. 219 women were diagnosed with lung cancer. The blood test was collected up to 6 months before the pathological examination, confirming the occurrence of lung cancer.

The LightGBM model had an AUC of 0.67, and the Ridge regression model had an AUC of 0.58.

### Example 4—Predicting Prostate Cancer Occurrence

In this fourth implementation example, the same models from example 1, trained on the breast cancer database and with NLR, RBC, and age as features, were tested to predict prostate cancer occurrence. To this end, blood test results were collected from 108 men aged 43 to 91. Fifty-six men were diagnosed with prostate cancer. The blood test results were collected up to two years before the pathological examination confirming the occurrence of prostate cancer.

The LightGBM model had an AUC of 0.54, and the Ridge regression model had an AUC of 0.66.

1. A method for training an artificial intelligence model to predict the chances of a solid tumor occurring in an individual, the method comprising at least the steps of:
receiving a first set of features, wherein the first set of features is related to individuals who have not been diagnosed with some type of solid tumor and comprises, as features for each individual, at least: age; at least one feature obtained from the red blood cell series of a blood count; and at least one feature obtained from the white blood cell series of a blood count;
receiving a second set of features, wherein the second set of features is related to individuals diagnosed with some type of solid tumor and comprises, as features for each individual, at least: age; at least one feature obtained from the red blood cell series of a blood count; and at least one feature obtained from the white blood cell series of a blood count; and
training the artificial intelligence model based on the first and second sets of features.

2. The method of claim 1, wherein the solid tumor to be predicted is selected from the group consisting of: breast cancer, lung cancer, colorectal cancer, prostate cancer, and ovarian cancer.

3. The method of claim 2, wherein the solid tumor is breast cancer.

4. The method of claim 1, wherein the first and second sets of features additionally comprise at least one feature obtained from the platelet series of a blood count.

5. The method of claim 1, wherein the first and second sets of features additionally comprise at least one feature obtained from the individual's phenotypic characteristics and/or from imaging tests or routine medical examinations.

6. The method of claim 1, wherein the model is trained using a supervised or unsupervised machine learning algorithm, or comprises the combination of models trained using a supervised and/or unsupervised learning algorithm.

7. A system for training an artificial intelligence model to predict the chances of a solid tumor occurring in an individual, the system comprising at least one processor, wherein the processor is configured to perform the method of claim 1.

8. A computer-readable medium comprising instructions that, when executed by at least one processor, cause the processor to perform the method of claim 1.

9. A method for predicting the chances of a solid tumor occurring in an individual, comprising the steps of:
receiving, as features for the individual, at least: age; at least one feature obtained from the red blood cell series of a blood count; and at least one feature obtained from the white blood cell series of a blood count; and
calculating, using a model trained according to the method of claim 1 and based on the received features, the chances of cancer occurring in the individual.

10. The method of claim 9, wherein the solid tumor to be predicted is selected from the group consisting of: breast cancer, lung cancer, colorectal cancer, prostate cancer, and ovarian cancer.

11. The method of claim 10, wherein the solid tumor is breast cancer.

12. The method of claim 9, wherein the first and second sets of features additionally comprise at least one feature obtained from the platelet series of a blood count.

13. The method of claim 9, wherein the first and second sets of features additionally comprise at least one feature obtained from the individual's phenotypic characteristics and/or from imaging tests or routine medical examinations.

14. A system for predicting the chances of a solid tumor occurring in an individual, the system comprising at least one processor, wherein the processor is configured to perform the method of claim 9.

15. A computer-readable medium comprising instructions that, when executed by at least one processor, cause the processor to perform the method of claim 9.
